(19) United States
(12) Patent Application Publication
(10) Pub. No.: US 2002/0165613 A1
(43) Pub. Date: Nov. 7, 2002

(54) Intervertebral fixation device having one or more bracing elements

(75) Inventors: Chih-I Lin, Chino Hills, CA (US); Seang Beng Tan, Singapore (SG); Hansen A. Yuan, Fayetteville, NY (US)

Correspondence Address: BACON & THOMAS, 625 Slaters Lane - 4th Floor, Alexandria, VA, 22314, US

(73) Assignee: Chih-I LIN, Chino Hills, CA

(21) Appl. No.: 10/134,525

(22) Filed: Apr. 30, 2002

(30) Foreign Application Priority Data
May 4, 2001 (TW) 90207298

(51) Int. Cl. A61F 2/44

(52) U.S. Cl. 623/17.11

(57) ABSTRACT

An intervertebral fixation device includes an intervertebral fixation block and one or more bracing elements. The fixation block is provided with one or more joining slots. The bracing elements support the vertebrae under treatment at an insertion front end thereof, when the bracing elements are inserted into the joining slots of the fixation block.

### FIELD OF THE INVENTION

The present invention relates generally to an intervertebral fixation device, and more particularly to an intervertebral fixation device which has one or more bracing elements.

### BACKGROUND OF THE INVENTION

The conventional intervertebral fixation device is generally defective in design in that it can not be adjusted in accordance with an interval or angle of the two adjacent vertebrae to be fixed, thereby resulting in a relatively poor fixation effect. Such a shortcoming of the conventional intervertebral fixation device as described above is due to the fact that the conventional intervertebral fixation device has a fixed shape and a fixed size.

### SUMMARY OF THE INVENTION

It is the primary objective of the present invention to provide an intervertebral fixation device with one or more bracing elements to facilitate the adjusting of interval and/or angle between two adjacent vertebrae under treatment. The present invention brings about a relatively good fixation effect on the vertebrae under treatment.

It is another objective of the present invention to provide an intervertebral fixation device which is formed of an intervertebral fixation block and a bracing element.

It is still another objective of the present invention to provide an intervertebral fixation device comprising an intervertebral fixation block, a bracing element, and a joining member.

An intervertebral fixation device constructed according to the present invention comprises:

an intervertebral fixation block provided with one or more joining slots; and

one or more bracing elements inserted into said one or more joining slots from one end of said intervertebral fixation block, so that an insertion front end of said one or more bracing elements protruding from said intervertebral fixation block for supporting a vertebra under treatment.

Preferably, said fixation block further comprises a joining part engageable with an externally-added joining member, so as to prevent said one or more bracing elements from slipping out of said one or more joining slots.

Preferably, said fixation block further comprises a joining part; wherein said one or more bracing elements have a joining member engageable with said joining part of said fixation block so as to prevent said bracing elements from slipping out of said one or more joining slots of said fixation block.

Preferably, said one or more joining slots of said fixation block are slanted; so that said one or more bracing elements gradually jutted out of said fixation block, when said one or more bracing elements are received in said one or more joining slots.

Preferably, said fixation block has two joining slots which are substantially coplanar.

Preferably, said one or more bracing elements have a blade side facing outwards from said one or more joining slots of said fixation block.

The features, functions and advantages of the present invention will be readily understood upon a thoughtful deliberation of the following detailed description of the preferred embodiments of the present invention with reference to the accompanying drawings.

### BRIEF DESCRIPTION OF THE DRAWINGS

FIGS. 1-a to 1-f are schematic views of the joining procedures of a first preferred embodiment of the present invention.

FIG. 2 shows a joining schematic view of a second preferred embodiment of the present invention.

FIGS. 3 and 4 are schematic views showing the joining relationship between the joining member and the intervertebral fixation block of a third preferred embodiment of the present invention.

### DETAILED DESCRIPTION OF THE INVENTION

The intervertebral fixation device of the present invention comprises an intervertebral fixation block and one or more bracing elements.

The intervertebral fixation block is provided with one or more joining slots. The insertion ends of the bracing elements brace the vertebrae under treatment via the joining slots of the intervertebral fixation block.

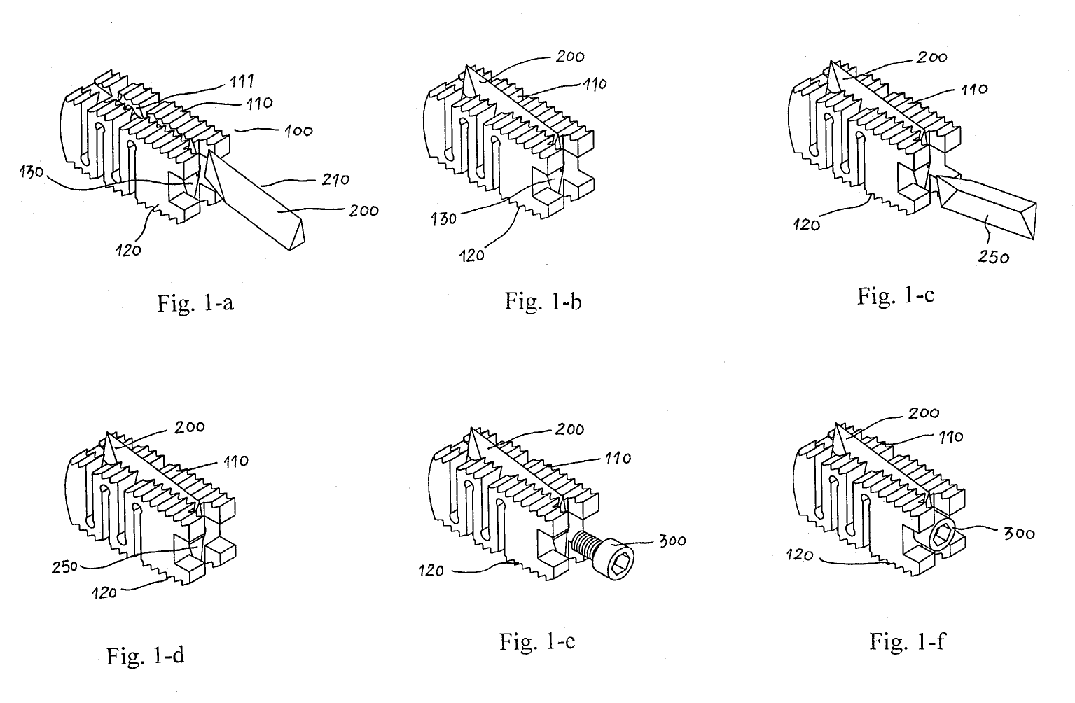

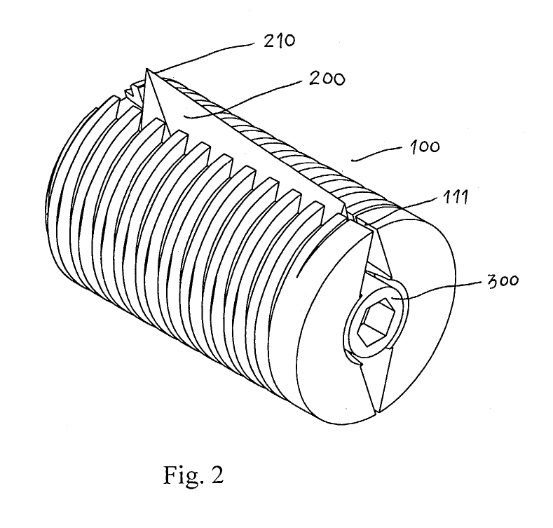

The intervertebral fixation block of the present invention is similar in construction to the conventional intervertebral fixation block, as shown in FIGS. 1 and 2. The intervertebral fixation block of the present invention has a smooth or rough outer surface, preferably the rough outer surface.

The intervertebral fixation block has preferably a plurality of joining slots to facilitate the inserting of the bracing elements. The ideal number of the joining slots is two. In the event that the fixation block is provided with only one joining slot, it is preferable that the joining slot faces upward (head) rather than downward (foot), thereby enabling the bracing element to be substantially perpendicular to the end plate of the vertebra such that the end plate is urged or fastened onto by the bracing element. If the fixation block is provided with two joining slots, these two joining slots should be substantially coplanar such that one faces upwards, and that other one faces downwards, thereby enabling the two bracing elements to engage in such a manner that they urge or fasten onto substantially vertically the lower end plate of the upper vertebra to be fixed and the upper end plate of the lower vertebra to be fixed. The two joining slots form an acute angle whose dividing line is preferably and substantially parallel to the end plate.

The bracing element of the present invention has any shape, preferably a barlike object with a blade side which must face the end plate at the time when the bracing element is inserted into the joining slot. The bracing element is preferably corresponding in shape to the joining slot of the fixation block, thereby enabling the bracing element to be fixed in the joining slot.

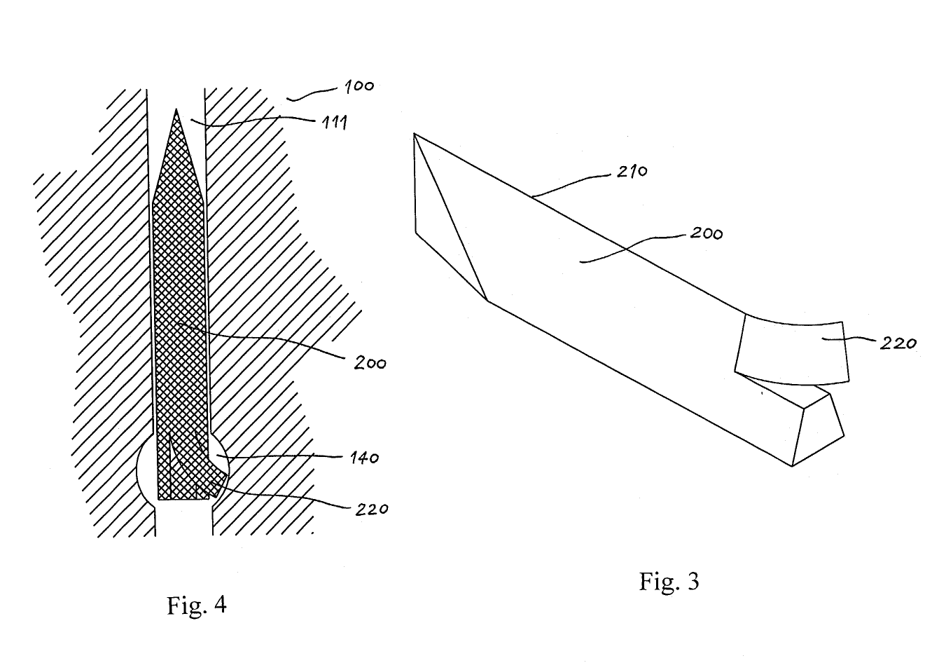

In order to prevent the escape of the bracing element from the joining slot into which the bracing element is inserted, the fixation block or bracing element may be provided at one end thereof with a joining part, so as to enable the joining part of the fixation block to join with the externally-added joining member, or to enable the joining part of the bracing element to join with the externally-added joining member, or to enable the joining part of the bracing element to join with the joining part of the fixation block, or to enable other complicated joining methods to be carried out. The joining methods are similar to the conventional methods, such as the use of threads, and shown in FIGS. 1 and 2, the use of retainers, as shown in FIGS. 3 and 4, or the use of mortise and tenon.

As shown in FIGS. 1a-1f, the intervertebral fixation device of the present invention comprises a fixation block 100 and two bracing elements 200 and 250. The fixation block 100 has two contact surfaces 110 and 120 for making contact with the end plates of the vertebrae to be fixed. The contact surface 110 is provided with a joining slot 111. A threaded hole is provided as a joining part at a location of 130. The bracing element 200 has a blade side 210. In the surgical operation, the fixation block is implanted and located, as shown in FIG. 1a, before the bracing element 200 is inserted into the joining slot 110, such that the blade side 210 of the bracing element 200 urges slantingly and upwardly the end plate of the vertebra to be fixed, as illustrated in FIG. 1b. Similarly, the second bracing element 250, as shown in FIGS. 1c and 1d, is inserted in the same manner into another joining slot. An externally-added screw 300 is then engaged with the threaded hole of the fixation block, as shown in FIGS. 1e and 1f.

As shown in FIG. 2, the intervertebral fixation block 100 of the second preferred embodiment of the present invention is of a cylindrical construction, wherein similar parts as shown in FIGS. 1a-1f are represented by the same numerals used therein.

As shown in FIGS. 3 and 4, the third preferred embodiment of the present invention is basically similar in construction to the first preferred embodiment described above with reference to FIG. 1, with the difference being that the bracing element 200 of the third preferred embodiment is provided at the tail end with a pre-curved tail end piece 220. As the tail end piece 220 is plugged into the joining slot 111 of the fixation block, the tail end piece 220 is exerted on by a force such that the tail end piece 220 contracts to the longitudinal axis of the bracing element 200. As the tail end piece 220 enters the round hole 140 of the joining slot 111 of the fixation block, the curved tail end piece 220 returns to its original shape to be retained in the round hole 140, thereby preventing the bracing element 200 from slipping out of the joining slot 111.

The embodiments described above are to be regarded in all respects as being merely illustrative and not restrictive. Accordingly, the present invention may be embodied in other specific forms without deviating from the spirit thereof. For example, the bracing element may be provided at the top end (other end of the curved tail end piece as shown in FIG. 3) with a tenon, whereas the joining slot is provided with a mortise corresponding in location to and engageable with the tenon. As the tenon is retained by the mortise, the bracing element can not slip out of the joining slot. In addition, the bracing elements of the present invention may be of a triangular columnar construction having different height. Depending on the need of the bracing angle between two adjacent vertebrae, the surgeon is provided with the bracing elements of various height.

What is claimed is:

1. An intervertebral fixation device comprising:
an intervertebral fixation block provided with one or more joining slots; and
one or more bracing elements inserted into said one or more joining slots from one end of said intervertebral fixation block, so that an insertion front end of said one or more bracing elements protruding from said intervertebral fixation block for supporting a vertebra under treatment.

2. The intervertebral fixation device as defined in claim 1, wherein said fixation block further comprises a joining part engageable with an externally-added joining member, so as to prevent said one or more bracing elements from slipping out of said one or more joining slots.

3. The intervertebral fixation device as defined in claim 1, wherein said fixation block further comprises a joining part; wherein said one or more bracing elements have a joining member engageable with said joining part of said fixation block so as to prevent said bracing elements from slipping out of said one or more joining slots of said fixation block.

4. The intervertebral fixation device as defined in claim 1, wherein said one or more joining slots of said fixation block are slanted; so that said one or more bracing elements gradually jutted out of said fixation block, when said one or more bracing elements are received in said one or more joining slots.

5. The intervertebral fixation device as defined in claim 1, wherein said fixation block has two joining slots which are substantially coplanar.

6. The intervertebral fixation device as defined in claim 4, wherein said fixation block has two joining slots which are substantially coplanar.

7. The intervertebral fixation device as defined in claim 1, wherein said one or more bracing elements have a blade side facing outwards from said one or more joining slots of said fixation block.

8. The intervertebral fixation device as defined in claim 4, wherein said one or more bracing elements have a blade side facing outwards from said one or more joining slots of said fixation block.

9. The intervertebral fixation device as defined in claim 5, wherein said one or more bracing elements have a blade side facing outwards from said one or more joining slots of said fixation block.

10. The intervertebral fixation device as defined in claim 6, wherein said one or more bracing elements have a blade side facing outwards from said one or more joining slots of said fixation block.
